(19) United States
(12) Patent Application Publication
(10) Pub. No.: US 2024/0131043 A1
(43) Pub. Date: Apr. 25, 2024

(54) SPIRAL STEROIDS AND PRECURSORS THEREOF FOR CLINICAL DIAGNOSIS AND TREATMENT OF NECROTIZING ENTEROCOLITIS AND PRE-ECLAMPSIA

(71) Applicant: Coddle Creek Capital, LLC, Kannapolis, NC (US)

(72) Inventor: Fred Chasalow, Belmont, CA (US)

(21) Appl. No.: 18/482,651

(22) Filed: Oct. 6, 2023

Related U.S. Application Data
(60) Provisional application No. 63/378,840, filed on Oct. 7, 2022.

(51) Int. Cl. A61K 31/665; A61K 33/00; A61K 47/68; C07K 16/00

(52) U.S. Cl. CPC A61K 31/665; A61K 33/00; A61K 47/6835; C07K 16/00

(57) ABSTRACT

The present disclosure relates to the use of certain steroids for the diagnosis and treatment of necrotizing enterocolitis and pre-eclampsia.

This application claims the benefit of U.S. Provisional Application No. 63/378,840, filed on Oct. 7, 2022, the entire contents of which are hereby incorporated by reference.

### BACKGROUND OF THE INVENTION

Necrotizing enterocolitis (NEC) is a serious condition that can affect newborn babies, where tissue in the bowel (small and large intestines) becomes inflamed. NEC is also described as an inflammation of the gut. Babies born premature or sick have more immature bowels, which means that they are more likely to get NEC than babies born healthy or term. NEC can affect just a small part of the bowel, or sometimes the whole bowel can be affected.

NEC usually affects newborn babies in the first few weeks after birth. It is more common in premature babies (particularly babies born before 32 weeks or who weigh less than 1500 g), babies having other medical problems, and babies who have been unwell before they were born (for example, babies who are not growing in the womb as expected).

The symptoms of NEC can develop over a period of days or appear suddenly. Commonly reported symptoms include:
Poor tolerance to feeding (not being able to digest food),
Bloating or swelling of the stomach (abdominal distention),
Stomach discoloration, usually bluish or reddish,
Pain when someone touches the abdomen,
Blood in the stools or a change in their volume or frequency,
Diarrhea, with change in the color and consistency of the stool, often containing frank (visible) blood,
Decreased activity (lethargy),
Vomiting greenish-yellow liquid,
Inability to maintain normal temperature, and
Episodes of low heart rate or apnea, a temporary stop in breathing.

In advanced cases, the blood pressure may drop and the pulse may become weak. Infants may develop fluid in the abdominal cavity or infection of the tissue lining the stomach (a condition called peritonitis), or they could go into shock. The affected area of the intestine may develop a hole or perforation in the wall requiring emergency surgery. Pressure from the abdomen can cause severe difficulty in breathing. In this case, the infant may need support from a breathing machine, or respirator.

Pre-eclampsia is a serious blood pressure condition that develops sometimes during pregnancy. Diagnosis of pre-eclampsia is based on hypertension, proteinuria (high levels of protein in the urea) and hypokalemia at 20 weeks of gestational age. Pre-eclampsia is dangerous for both the mother and her developing fetus. Mothers who had pre-eclampsia and their babies have a two- to four-fold lifetime increased risk of cardiovascular and end-stage renal diseases.

Pre-eclampsia complicates up to 8% of all deliveries worldwide. In the United States, it is the cause of about 15% of premature deliveries (delivery before 37 weeks of pregnancy).

Signs of pre-eclampsia include:
Headaches and seizures,
Blurry vision or light sensitivity,
Dark spots appearing in your vision,
Right side abdominal pain,
Swelling in the hands and face (edema), and
Shortness of breath.

There is a need for new treatments for necrotizing enterocolitis and pre-eclampsia.

### SUMMARY OF THE INVENTION

The present inventor has surprisingly found that a newly discovered class of steroids, characterized by a spiral steroid, are surprisingly effective for the diagnosis and treatment of necrotizing enterocolitis (NEC) and pre-eclampsia. The steroids described herein (i) may have 23, 24 or 25 carbon atoms, (ii) are phosphodiesters, and (iii) contain a spiral steroid at carbon #17, or are a precursor thereof.

Accordingly, in one aspect, the present invention relates to a method of treating necrotizing enterocolitis in an infant baby or preventing, inhibiting, or suppressing necrotizing enterocolitis in an infant baby at risk thereof. The method comprises administering to the infant baby, or to the maternal patient prior to delivery (pregnant patient), a therapeutically effective amount of steroid C381 (galotropin™), steroid E381, a precursor thereof, a pharmaceutically acceptable salt thereof, or any combination of any of the foregoing. In one embodiment, the blood (serum) potassium levels are monitored in the infant baby to evaluate the efficacy of the treatment, and the amount and/or frequency of the administration of the steroid, such as C381 or E381, is modified accordingly.

Another aspect is a method of preventing, inhibiting, or suppressing necrotizing enterocolitis in a baby in the womb of a pregnant woman comprising administering to the pregnant woman a therapeutically effective amount of steroid C381 (galotropin™), steroid E381, a precursor thereof, a pharmaceutically acceptable salt thereof, or any combination of any of the foregoing.

In yet another aspect, the present invention relates to a method of diagnosing the risk of necrotizing enterocolitis in an infant baby. The method comprises testing a body fluid sample, from the pregnant (maternal) patient (e.g., prior to birth of the baby, such as in the last trimester) or from the infant baby, to determine the amount of steroid C381 (galotropin™), steroid E381, a precursor thereof, or any combination thereof. The method may further comprise comparing the amount of steroid C381, steroid E381, precursor, or any combination of any of the foregoing in the infant baby to the normal levels in an infant baby of similar age, and where the levels of steroid C381 or steroid E381 are below the normal levels (e.g., at least 25%, 40%, 50%, 60%, or 75% below normal levels) assessing the infant baby as at risk for NEC.

In another aspect, the present invention relates to a method for treating pre-eclampsia in a pregnant patient (maternal patient) or preventing worsening pre-eclampsia in a pregnant patient. The method comprises administering to the pregnant patient a therapeutically effective amount of steroid C369 (kaleotropin™) or a precursor thereof (such as E369, C353 or E353), a pharmaceutically acceptable salt thereof, or any combination of any of the foregoing.

In the methods described herein, the steroid, such as C381 and E381, may be administered in the form of a pharmaceutical composition, such as parenteral liquid composition or an oral composition, such as a liquid or solid dosage form (e.g., a tablet or capsule).

In another aspect, the present invention relates to a method of diagnosing a pregnant (maternal) patient as at risk for pre-eclampsia. The method comprises measuring the amount of steroid C313, steroid C329, steroid C341, a precursor thereof, or any combination thereof in the blood serum of the maternal patient. The method may further comprise comparing the amount of steroid C381, steroid E381, a precursor thereof, or any combination of any of the foregoing in the maternal patient to the normal levels in a maternal patient of the same gestational age, and where the levels of steroid C381, steroid E381, a precursor thereof, or any combination of any of the foregoing are below the normal levels (e.g., at least about 25%, about 40%, about 50%, about 60%, or about 75% below normal levels) assessing the maternal patient as at risk for pre-eclampsia.

In yet another aspect, the present invention relates to a method of diagnosing worsening pre-eclampsia in a maternal patient having pre-eclampsia. The method comprises measuring the amount of steroid C313, steroid C329, steroid C341 (ionotropin™), a precursor thereof, or any combination thereof in the blood serum of a maternal patient with pre-eclampsia. The method may further comprise comparing the amount of steroid C381, steroid E381, a precursor thereof, or any combination of any of the foregoing in the maternal patient to the normal levels in a maternal patient, and where the levels of steroid C381, steroid E381, a precursor thereof, or any combination of any of the foregoing are below the normal levels (e.g., at least 25%, 40%, 50%, 60%, or 75% below normal levels) assessing the maternal patient as having worsening pre-eclampsia.

In yet another aspect, the present invention relates to a method of treating hypokalemia in a pregnant (maternal) patient. The method comprises administering to the maternal patient a therapeutically effective amount of steroid C369 (kaleotropin™) or a precursor thereof (such as E369), a pharmaceutically acceptable salt thereof, or any combination of any of the foregoing.

In another aspect, the present invention relates to a method of making an antibody against any of steroid compounds C381, E381, C341, C313, C329, and C369.

Yet another aspect is an antibody against any of steroid compounds C381, E381, C341, C313, C329, and C369.

In another aspect, the present invention relates to a method for treating seizures in a maternal (pregnant) patient with pre-eclampsia. The method comprises administering a therapeutically effective amount of a monoclonal antibody to steroid C341.

Yet another aspect is an infant formula or baby food supplemented with steroid C381, steroid E381, a pharmaceutically acceptable salt thereof, or any combination of any of the foregoing (for example, a therapeutically effective amount, for example, to prevent, inhibit, or suppress NEC). In one embodiment, the infant formula or baby food further comprises potassium, sodium, calcium, or any combination thereof. In one embodiment, the infant formula or baby food further comprises potassium. In one embodiment, the infant formula or baby food is a starter formula or food. In another embodiment, the infant formula or baby food is a follow-on formula or food. In one embodiment, the steroid is added to the infant formula or baby food after pasteurization of any milk in the formula.

Yet another aspect is method of preparing monoclonal and/or polyclonal antibodies, the method comprising (a) isolating a specific steroid-phosphoethanolamine diester, and (b) preparing monoclonal and/or polyclonal antibodies using the diester as a hapten. In one embodiment, step (b) comprises (i) conjugating a polypeptide to the diester to form an immunogenic composition, (ii) immunizing an animal with the immunogenic composition, and collecting monoclonal and/or polyclonal antibodies from the animal (e.g., the serum of the animal). In one embodiment, the steroid-phosphoethanolamine diester is E381 and the antibodies are selected for C381. In another embodiment, the diester is selected from E313, E329, E341, E353, E369, E371, E381, or another steroid phosphoethanolamine diester, and the antibodies are selected for the corresponding steroid phosphocholine diester and optionally used for specific immunoassays (e.g., detecting a steroid phosphocholine diester in an immunoassay with the antibodies).

### DETAILED DESCRIPTION OF THE INVENTION

### Definitions

As used herein, the term “spiral steroid” means a steroid compound having the steroid core structure of seventeen carbon atoms, bonded in four “fused” rings (six-membered rings A, B, and C, and a five-membered ring D), with an additional ring E attached to ring D via a spiral configuration at carbon #17. The planar axes of rings D and E are perpendicular (i.e., rings D and E are not fused and share carbon #17). The spiral steroids described herein are phosphodiesters. In the nomenclature of the spiral steroids described herein, the prefix “C” designates phospho-choline as the phosphodiester and the prefix “E” designates phospho-ethanolamine as the phosphodiester. Examples of spiral steroids that may be used in the methods described herein include C381 and E381. The structures of several steroids, including spiral steroids C381 and E381, are shown below. See also Chasalow, Int. J. Mol. Sci., “An Introduction to Spiral Steroids,” 2022, 23 (17), 9523, https//doi.org/10.3390/ijms23179523, which is hereby incorporated by reference.

The chemical structures of the steroid compounds referenced herein are shown below.

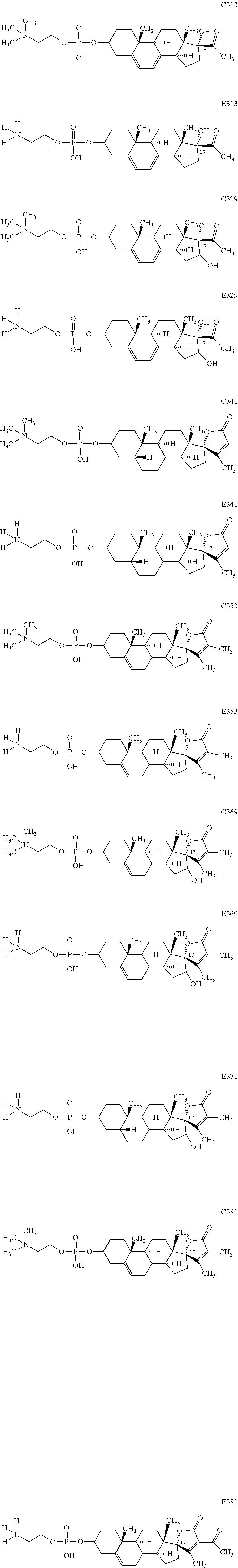

As used herein, the term “about” when referring to a number or a numerical range means that the number or numerical range referred to is an approximation within experimental variability (or within statistical experimental error), and thus the number or numerical range may vary from, for example, between 1% and 15% of the stated number or numerical range.

As used herein, the term “comprising” (and related terms such as “comprise” or “comprises” or “having” or “including”) includes, but is not limited to, those embodiments, for example, an embodiment of any composition of matter, composition, method, or process, or the like, that “consist of” or “consist essentially of” the described features.

As used herein, the term “effective amount” or “therapeutically effective amount” refers to that amount of a compound described herein that is sufficient to effect the intended application including, but not limited to, disease treatment. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on the particular compounds chosen, the dosing regimen to be followed, whether it is administered in combination with other compounds, timing of administration, the tissue to which it is administered, and the physical delivery system in which it is carried.

As used herein, the terms “treatment” and “treating” refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A “therapeutic effect,” as that term is used herein encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “subject” or “patient” refers to an animal, such as a mammal, for example a human. In some embodiments, the patient is a human, and in some embodiments, the patient is human infant. In some embodiments, the patient is a maternal human.

The term “infant formulas” refers to formulas which cover the nutrition need of an infant. The infant formula may contain protein, carbohydrate, fat, or any combination of any of the foregoing.

The term “starter formulas” refers to formulas for babies which cover their nutrition needs within the first 4-6 months. The starter formula may contain protein, carbohydrate, fat, or any combination of any of the foregoing.

The term “follow-on formulas” refers to formulas for babies which cover their nutrition needs after the first 4 months. The follow-on formula may contain protein, carbohydrate, fat, or any combination of any of the foregoing.

As used herein, the term “infant” means a baby from birth to the third month. The term “infant” also encompasses the term “preterm infant”, which means a baby born before 37 completed weeks of gestation. In additional embodiments, the preterm infant is born at less than 30 weeks of gestational age or has a birthweight under 1.5 kg.

In additional embodiments of any of the methods described herein, the preterm infant was delivered in the 8^{th} month or earlier of pregnancy, such as in the 7^{th} month of pregnancy or the 8^{th} month of pregnancy.

In some embodiments of any of the methods described herein, the infant is a preterm infant. In some embodiments, the infant is being (at that present time and previously) fed by an enteral route such as gavage or tube feedings. In some embodiments, the infant's mother had pre-eclampsia during the pregnancy (which, e.g., makes the infant baby at risk of developing necrotizing enterocolitis).

As used herein, the term “antibody” refers to an immunoglobulin, whether produced naturally or synthetically (e.g., recombinant), either in whole or in part. The term “antibody” also encompasses antibody fragments, which refers to any derivative of an antibody that is less than full length while retaining at least a portion of the full-length antibody's specific binding ability. Examples of antibody fragments include, e.g., Fab, Fab′, F(ab)2, F(ab′)2, Fv, dsFv, single chain Fab (scFab), single-chain Fvs (scFv), diabodies (dimeric scFv), and bispecific antibodies. The fragment can include multiple chains linked together. The term ‘antibody’ also encompasses other antibody-like formats such as nanobodies and minibodies. See, e.g., Bates et al., “David vs. Goliath: The Structure, Function, and Clinical Prospects of Antibody Fragments” (2019) Antibodies, 8:28; and Roland Kontermann, “Alternative antibody formats” (2010) Current Opinion in Molecular Therapeutics, 12 (2): 176-83.

In utero, potassium is delivered through the blood supply. Post-delivery, the gastrointestinal tract needs to start collecting potassium, and to mature and double in size during the first two weeks. Without potassium, this does not happen. Paneth cells appear to have a major role in the growth and development of the gastrointestinal tract. See, e.g., Lueschow et al., Front. Immunol., 11, April 2020, doi:10.3389/fimmu.2020.00587. Without wishing to be bound by theory, the inventors theorize that (i) galotropin™ is made in the placenta, (ii) galotropin™ interacts with the NaK-ATPase in the gastrointestinal tract during the 9^{th} month of gestation and the NaK-ATPase has the alpha-4 subunit, (iii) the absence of galotropin™ leads to an immature gastrointestinal tract and neonatal hypokalemia, (iv) after delivery, the source of galotropin™ is gone leading to no stimulation of the NaK-ATPase, (v) it takes about 2 weeks to re-configure the gastrointestinal tract to synthesize the alpha-1 subunit, (vi) this reconfiguration process requires potassium (K⁺) and does not start if the infant has hypokalemia, and (vii) as a result, the infant can develop NEC because the gastrointestinal cells are unable to stretch to form tight junctions.

In embodiments of any of the methods described herein, a therapeutically effective amount of a steroid (such as, e.g., C381, E381, C369 and/or E369) is administered.

In embodiments of any of the methods described herein, the steroids and compositions described herein can be administered by any route that enables delivery of the compound(s) (e.g., steroids) to the site of action, such as orally, enterally, intranasally, topically (e.g., transdermally), intraduodenally, parenterally (including intravenously, intraarterially, intramuscularally, intravascularally, intraperitoneally or by injection or infusion), intradermally, by intramammary, intrathecally, intraocularly, retrobulbarly, intrapulmonary (e.g., aerosolized drugs) or subcutaneously (including depot administration for long term release e.g., embedded-under the-splenic capsule, brain, or in the cornea), sublingually, anally, rectally, vaginally, or by surgical implantation (e.g., embedded under the splenic capsule, brain, or in the cornea).

For oral or enteral administration, the steroid (such as, but not limited to, C381 or E381, or a precursor thereof) may be an additive ingredient of an infant nutrition given to the infant, such as, e.g., infant formula, baby food, formula milk, breast milk, solid or semisolid food, water, or a nutritional fluid.

Potassium may be administered in combination with the steroid (for example, as an additional additive in the infant nutrition, infant formula, or baby food). In one embodiment, oral administration of potassium is provided. The potassium may be in the same infant formula or baby food as the steroid C381 or E381.

In certain embodiments of any of the methods described herein, the clinical efficacy of the steroid (such as C381 or E381, or a precursor thereof) is determined by analyzing, e.g., resolution of necrotizing enterocolitis, reduced abdominal symptoms, reduced body temperature, reduced diarrhea, reduced vomiting, and/or improved appetite.

The amount of the steroid (such as C381, E381, a precursor thereof, or any combination of any of the foregoing) to be administered is dependent on the subject being treated, the severity of the disorder or condition, the rate of administration, the disposition of the compound and the discretion of the prescribing physician. An effective dosage may be in the range of about 0.001 to about 100 mg per kg body weight per day, such as about 0.01 to about 1 mg per kg body weight per day or about 0.01 to about 5 mg per kg body weight per day, in single or divided doses.

In additional embodiments of any of the methods described herein, the steroid (such as C381 or E381, or a precursor thereof) is administered in a concentration of about 0.01 to about 50 mg/L, such as about 0.1 to about 1 mg/L.

An effective amount of a compound (e.g., steroid, such as C381 or E381) of the present invention may be administered in either single or multiple doses (e.g., one, two, three of four times a day).

### Necrotizing Enterocolitis

In some embodiments, the method comprises diagnosing the risk of necrotizing enterocolitis in an infant baby, as described herein. This may be useful in guiding the procedures for treating or preventing necrotizing enterocolitis in the infant baby. In some embodiments, the method comprises obtaining a blood sample from the infant baby and assaying the blood sample (serum) for the amount of potassium. Monitoring the infant's blood (serum) potassium levels may be useful for gauging the effectiveness of the treatment and modifying the administration of the steroid such as C381 or E381, or a precursor thereof) to the infant baby accordingly (such as changing the dose amount, changing the dosing frequency).

Another aspect of the present invention is a method of diagnosing the risk of necrotizing enterocolitis in an infant baby by testing the maternal serum (during pregnancy, such as the last six weeks of pregnancy), maternal milk (such as colostrum), cord blood serum (from the birth of the infant), or the infant's serum for steroid C381 or C369. The method comprises obtaining a body fluid sample. Examples of suitable body fluids include, e.g., maternal blood (serum), placental blood (serum), maternal breast milk (e.g., colostrum), or infant blood (serum). The method comprises assaying the sample for the amount of steroid C381 or C369 therein (e.g. concentration levels). In some embodiments, this risk diagnosis is performed in a maternal patient who has pre-eclampsia, which is a risk factor for the infant developing NEC. If the amount of steroid C381 or C369 is considered too low, then the infant or maternal patient could be treated with steroid C381 or C369 in the manner described herein.

The timing for administering steroid C381 to the maternal patient may be an important factor. For example, it may be important to administer the steroid to the maternal patient as soon as a physician recognizes the impending need for C-section delivery of the infant. Thus, in some embodiments, steroid C381 is administered to the maternal patient at a time within about 12, 8, 4, or 2 hours of a C-section delivery. In this setting, giving the steroid in association with a C-section delivery provides the maternal patient with a higher level of steroid C381, for example, comparable to what she would have made herself if she was instead having a normal full-term delivery.

In some embodiments, the method further comprises determining the relationship between the amount of steroid C381 in the body fluid and the development of necrotizing enterocolitis (e.g., onset of symptoms thereof, severity of symptoms, timing of resolution, or response to steroid C381 treatment).

In some embodiments, the method further comprises obtaining a body fluid sample from the infant baby (e.g. blood serum) or maternal patient (e.g. breast milk), and assaying the sample for the amount of potassium. A low potassium level may be useful for augmenting the diagnosis on the risk of developing necrotizing enterocolitis.

Another aspect of the present invention is a method of preventing necrotizing enterocolitis in an infant by treating the maternal patient with steroid C381 or C369 or a pharmaceutically acceptable salt thereof. The method comprises administering a therapeutically effective amount of steroid C381 or C369 or a pharmaceutically acceptable salt thereof to the maternal patient.

In some embodiments, the method further comprises determining the relationship between the amount of steroid C381 given to the maternal patient and clinical efficacy (e.g. reduced incidence of necrotizing enterocolitis, normalized serum potassium levels, or normalized blood pressure).

In some embodiments, the method further comprises obtaining a blood sample from the maternal patient and assaying the blood sample (serum) for the amount of potassium. The maternal patient's potassium level may be useful for guiding administration of steroid C381 thereto accordingly (such as changing dose amount or changing dosing frequency).

Steroid E381 is an analogue (precursor) of steroid C381. Thus, for any of the methods described herein, steroid E381 could be used instead of C381, or in combination therewith, for the relevant C381 related methods described herein.

### Pre-Eclampsia

Another aspect of the present invention is a method for treating pre-eclampsia in a maternal patient, or prevention of pre-eclampsia in a maternal patient who is at risk thereof. The method comprises administering a therapeutically effective amount of steroid C369, C353 or C329 to the maternal patient.

In some embodiments, the method further comprises determining the relationship between the amount of steroid C369, C353 or C329 given to the maternal patient and clinical efficacy (e.g., resolution of hypertension, or normalized serum potassium levels).

In some embodiments, the method further comprises guiding the treatment based on the method described below for making a prediction of impending worsening pre-eclampsia. The method comprises assaying the maternal blood sample (serum) for the amount of steroid C313, steroid C329, or steroid C341 to perform the diagnostic method described below. The maternal patient's blood levels of these steroids may be useful for guiding administration of steroid C369 or C329 thereto accordingly (such as changing the dose amount or changing the dosing frequency).

In some embodiments, the method further comprises assaying the blood sample (serum) for the amount of potassium. The maternal patient's potassium level may be useful for guiding administration of steroid C369 or C329 thereto accordingly (such as changing the dose amount or changing the dosing frequency).

Another aspect of the present invention is a method of diagnosing worsening pre-eclampsia in a maternal patient with pre-eclampsia. The method comprises obtaining a blood sample from the maternal patient. In some embodiments, the blood sample is obtained at about 10 weeks of gestation or later (e.g., 12, 15, 18, 20, 22, 24, or 26 weeks or later). The blood sample may be analyzed to assay the amount of steroid C313, steroid C329, or steroid C341 in the blood sample, which may then be compared to an applicable reference criteria (as discussed herein). If the amount of steroid C313, steroid C329, or steroid C341 in the blood sample meets or exceeds the applicable reference criteria, a diagnosis of worsening pre-eclampsia may be made.

It has been found that C313 serum levels are 0.7 μg/ml in pregnant women with normal blood pressure, compared to 1.5 μg/ml in pregnant women with hypertensive pre-eclampsia. Accordingly, we propose a threshold value X as a reference criteria, where a blood serum level of X or greater indicates a diagnosis of worsening pre-eclampsia. Based on the findings, the value for X could be in the range of about 1.0 μg/ml or greater; such as about 1.2 μg/ml or greater. For example, X could be designated as 1 about 0.3 μg/ml such that a serum level of about 1.3 μg/ml or higher would indicate a diagnosis of worsening pre-eclampsia. A maximum value for X could be designated as about 10 μg/ml.

Because steroid C313 converts to C341 in vivo, detecting elevated levels of C341 is also be useful. It has been found that C341 serum levels are 0.2 μg/ml in pregnant women with normal blood pressure, compared to 0.5 μg/ml in pregnant women with hypertensive pre-eclampsia. Accordingly, a blood serum level of a reference criteria or greater (e.g., about 0.2 μg/ml or greater, about 0.3 μg/ml or greater, about 0.4 μg/ml or greater, or about 0.5 μg/ml or greater) indicates a diagnosis of worsening pre-eclampsia. For example, the threshold level could be designated as about 0.5 μg/ml such that a serum level of about 0.5 μg/ml or higher would indicate a diagnosis of worsening pre-eclampsia. A maximum value could be designated as about 10 μg/ml.

C329 is a precursor for spiral steroid C369, in which high levels indicate a lower risk of life-threatening hypertension or seizures. Thus, measuring C329 could also be useful. It has been found that C329 serum levels are 0.7 μg/ml in pregnant women with normal blood pressure, compared to 1.3 μg/ml in pregnant women with hypertensive pre-eclampsia. Accordingly, a blood serum level of a reference criteria or greater (e.g., about 1.0 μg/ml or greater, about 1.2 μg/ml or greater, or about 1.3 μg/ml or greater) indicates a decreasing risk of life-threatening hypertension or seizures. For example, the threshold level could be designated as about 1.3 μg/ml. A maximum value could be designated as about 10 μg/ml.

In another embodiment, the present invention provides a method for treating worsening pre-eclampsia in a maternal patient, or the prevention of worsening thereof (preventing life-threatening hypertension) in a maternal patient with pre-eclampsia. The method comprises the step of selecting or modifying a treatment for the pre-eclampsia. In some cases, the treatment method comprises administering a therapeutically effective amount of steroid C329, steroid C369, or a pharmaceutically acceptable salt thereof to the maternal patient. Treatment with steroid C329, steroid C369, or a pharmaceutically acceptable salt thereof may result in reducing maternal blood levels of steroid C313, or its conversion metabolite C341.

In some embodiments, the method further comprises determining the relationship between amount of steroid C329 or In some embodiments, the method further comprises C369 given to the maternal patient and clinical efficacy (e.g. reduced blood pressure, normalized serum potassium levels, reduced proteinuria, reduced blood serum levels of C313 or its hypertension-inducing metabolite C341) without inducing hypertension or renal dysregulation.

### Clinical Laboratory Assay

The steroids described herein may be detected and quantified by any suitable technology known to one of ordinary skill in the art, such as, e.g., mass spectrometry (MS) or antibody immunoassay. Having a clinical antibody immunoassay could be particularly useful. Examples of such assays include, e.g., ELISA (enzyme-linked immunosorbent assay), fluorescent-tagged antibodies with fluorescence biosensors, LFIA (lateral flow immunoassay) and radioactive-labeled antibodies. One exemplary, non-limiting, immunoassay for, e.g., C313, would involve (i) preparing deuterated C313 by reacting E313 with CD₃-X (where X is a leaving group) to form deuterated C313, (ii) establishing a standard curve by mixing known amounts of authentic deuterated material (e.g., deuterated C313) and measuring the ratio, and (iii) repeating the measurement (e.g., by mass spectroscopy (MS)) with clinical samples and comparing to the standard curve.

As such, another aspect of the present invention is a method of making an antibody against any of the steroid compounds described herein being used as a diagnostic marker; the resulting antibodies themselves; and performing clinical diagnosis using such antibodies. Examples of such steroid compounds include C381, E381, C341, C313, C329, and C369. Suitable methods for making antibodies are known in the art.

Production of antibodies against the targeted steroid compound could be performed by any suitable method known to one of ordinary skill in the art, including traditional polyclonal or monoclonal techniques. For clinical diagnostic purposes, the antibodies should be highly specific and highly sensitive for the targeted steroid compound. Because the steroid compounds described herein are relatively smaller molecules, conjugating the targeted steroid compound to a carrier protein (resulting in a hapten) may be done for improved success. Because E381 has an amine group that can facilitate a coupling reaction, E381 may be a particularly good candidate for conjugating to a carrier protein (i.e. a hapten).

Multiple copies of the targeted steroid compound may be conjugated to the carrier protein (e.g., keyhole limpet hemocyanin and bovine serum albumin) to further improve success rates. Examples of techniques for finding antibodies include, e.g., hybridoma screen, phage display, display library, etc. See, e.g., Lu et al., “Frontier of therapeutic antibody discovery: The challenges and how to face them” (2012) World J Biol Chem, 3 (12): 187-196; and Valldorf et a.l, “Antibody display technologies: selecting the cream of the crop” (2022) Biological Chemistry, 403 (5-6):455-477. The antibodies could be monoclonal or made by recombinant techniques.

### Synthesis of Steroids

Steroid C381 (galotropin™) may be prepared as shown in the scheme below.

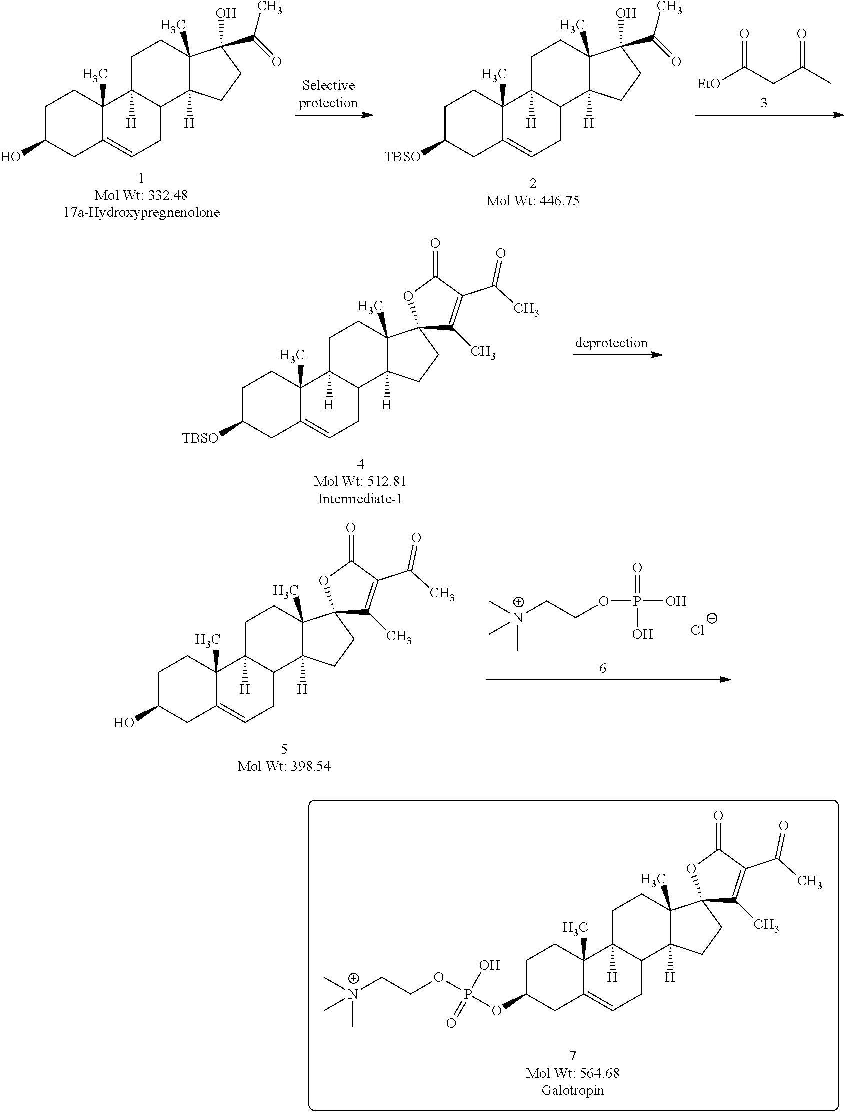

The hydroxyl group at the 3-position of 17α-hydroxypregnenolone 1 can be selectively protected using silyl protection groups (e.g., TBS (tert-butyl(dimethyl)silyl)) to obtain compound 2, which can be subjected to Spiro-cyclization in the presence of ethyl acetoacetate to afford compound 4, which can then be deprotected to obtain precursor compound 5. The choline group may then be attached to obtain steroid C381 (galotropin™) 7. The other steroid compounds described herein can be prepared by analogous methods.

### Baby Formulas Containing Steroid C381 (Galotropin™) or E381

In a further aspect, the present invention relates to a baby formula (aka infant formula) comprising a steroid as described herein (such as C381 or E381, or a precursor thereof). Suitable baby formulas are well known in the art. In one embodiment, the steroid is added after pasteurization of any milk in the formula. Such a baby formula may be used in any of the methods described herein.

The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A method of treating necrotizing enterocolitis in an infant baby in need thereof comprising administering a therapeutically effective amount of steroid C381 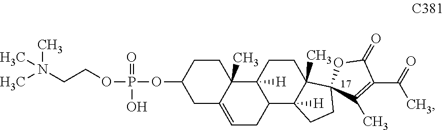
steroid E381 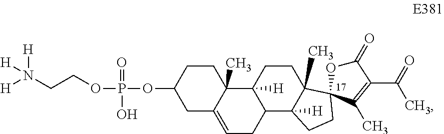
a pharmaceutically acceptable salt thereof, or any combination of any of the foregoing, to the infant baby.

2. A method of preventing necrotizing enterocolitis in an unborn infant baby at risk thereof of a pregnant patient comprising administering a therapeutically effective amount of steroid C381 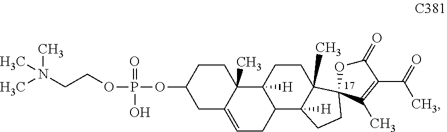
steroid E381 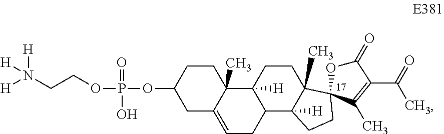
a pharmaceutically acceptable salt thereof, or any combination of any of the foregoing, to the pregnant patient prior to delivery of the infant baby.

3. A method for treating pre-eclampsia or preventing worsening pre-eclampsia in a pregnant patient comprising administering a therapeutically effective amount of steroid C369 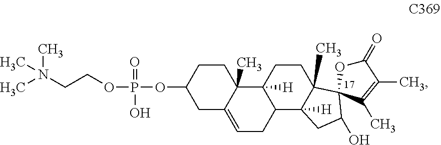
steroid E369 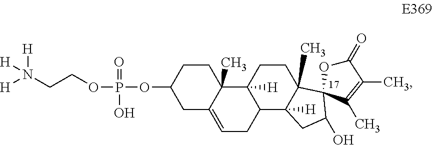
a pharmaceutically acceptable salt thereof, or any combination of any of the foregoing, to the pregnant patient.

4. The method of claim 1, wherein the infant baby is delivered in the 8^{th} month or earlier.

5. The method of claim 1, wherein the infant baby is a preterm infant born before 37 completed weeks of gestation.

6. The method of claim 1, wherein the method comprises administering about 0.01 to about 100 mg per kg body weight of steroid C381, steroid E381, a pharmaceutically acceptable salt thereof, or any combination of any of the foregoing per day.

7. The method of claim 1, wherein steroid C381, steroid E381, a pharmaceutically acceptable salt thereof, or any combination of any of the foregoing is administered as part of a baby formula.

8. The method of claim 1, further comprising administering potassium.

9. The method of claim 1, wherein steroid C381, steroid E381, a pharmaceutically acceptable salt thereof, or any combination of any of the foregoing is administered orally.

10. Infant formula or baby food supplemented with steroid C381 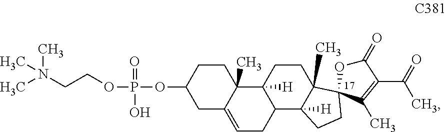
steroid E381 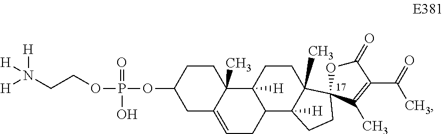
a pharmaceutically acceptable salt thereof, or any combination of any of the foregoing.

11. The infant formula or baby food of claim 10, further comprising potassium.

12. The infant formula or baby food of claim 10, wherein the infant formula or baby food is a starter formula or food.

13. The infant formula or baby food of claim 10, wherein the infant formula or baby food is a follow-on formula or food.

14. A method of preparing monoclonal and/or polyclonal antibodies, the method comprising (a) isolating a specific steroid-phosphoethanolamine diester, and (b) preparing monoclonal and/or polyclonal antibodies using the diester as a hapten.

15. The method of claim 14, wherein step (b) comprises (i) conjugating a polypeptide to the diester to form an immunogenic composition, (ii) immunizing an animal with the immunogenic composition, and collecting monoclonal and/or polyclonal antibodies from the animal.

16. The method of claim 14, wherein the steroid-phosphoethanolamine diester is E381 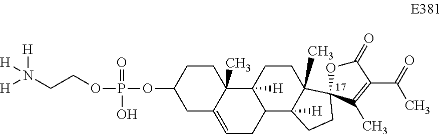
and the antibodies are selected for steroid C381

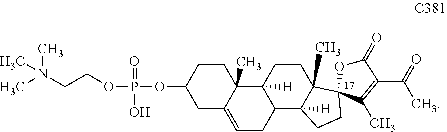

17. The method of claim 14, wherein the diester is selected from 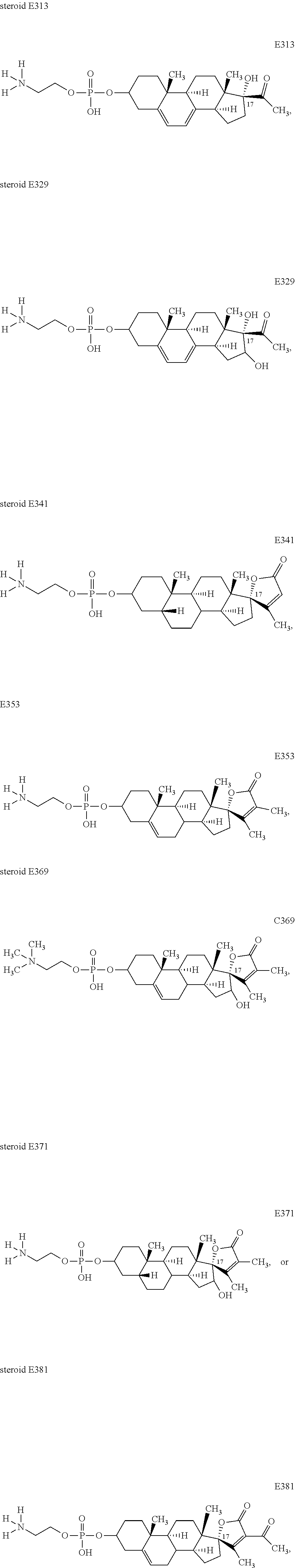
and the antibodies are selected for the corresponding steroid phosphocholine diester and optionally, detecting a steroid phosphocholine diester in an immunoassay with the antibodies.

18. A method of treating hypokalemia in a pregnant patient comprising administering to the patient a therapeutically effective amount of steroid C369 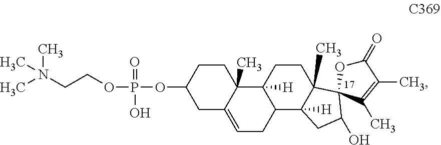
steroid E369 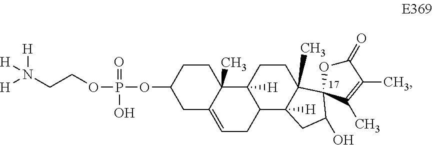
a pharmaceutically acceptable salt thereof, or any combination of any of the foregoing.

19. A method of identifying an infant baby at risk for necrotizing enterocolitis comprising (a) detecting the amount of steroid C381 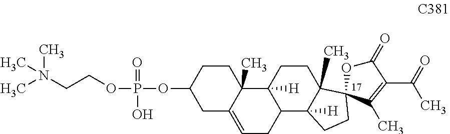
and/or steroid C369 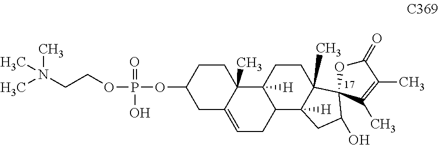
in a sample of (i) the serum of the pregnant mother to the infant baby, (ii) colostrum of the mother to the infant baby, (iii) cord blood serum of the infant baby, or (iv) the serum of the infant baby, and (b) where the amount is below the normal level of C381 and/or C369 in such a sample, identifying the infant baby as at risk for necrotizing enterocolitis.

20. The method of claim 19, further comprising step (c) administering a therapeutically effective amount of steroid C381 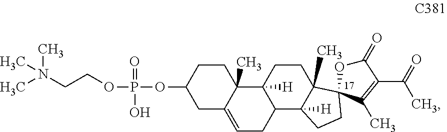
steroid E381 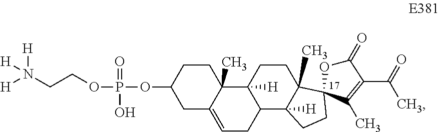
a pharmaceutically acceptable salt thereof, or any combination of any of the foregoing, to the infant baby to treat, prevent, inhibit, or suppress necrotizing enterocolitis in the infant baby.
